(19) United States
(12) Patent Application Publication
(10) Pub. No.: US 2007/0154415 A1
(43) Pub. Date: Jul. 5, 2007

(54) Inorganic light-absorbing micropigments and use thereof

(75) Inventors: Gerd Dahms, Duisburg (DE); Holger Seidel, Duisburg (DE); Olli-Pekka Antinluoma, Pori (FI)

Correspondence Address: BIRCH STEWART KOLASCH & BIRCH, PO BOX 747, FALLS CHURCH, VA, 22040-0747, US

(73) Assignee: Kemira Pigments Oy, Pori (FI)

(21) Appl. No.: 10/557,180

(22) PCT Filed: May 22, 2003
(86) PCT No.: PCT/EP03/05367

§ 371 Date: Oct. 10, 2006

(30) Foreign Application Priority Data
Mar. 19, 2003 (DE) 203-07-773.3

(51) Int. Cl. A61K 8/29; B32B 1/00; C04B 14/00; A61K 8/28; A61K 8/27

(52) U.S. Cl. 424/59; 428/403; 428/407; 106/401; 977/926

(57) ABSTRACT

Inorganic light-absorbing micropigments to which aluminium phosphates are applied are used in sunscreens or cosmetics emulsions.

The invention relates to the use of inorganic light-absorbing micropigments in sunscreens or cosmetics emulsions, suitable inorganic light-absorbing pigments or micropigments, and a process for production thereof.

For a long time, sunscreens and cosmetics emulsions have been known which comprise micronized titanium dioxide as micropigment. In addition to organic light-protection filters, the micronized or microfine titanium dioxide serves to absorb and to reflect light. Customary applications involve, in particular, UV-A and UV-B protection by sunscreens which also have corresponding UV-A and UV-B absorbing action.

Owing to the increasing UV exposure of human skin, there is an increasing demand for more effective sunscreens which cause improved UV-A and UV-B absorption. The sunscreens contain UV absorbers or light filters which generally convert the UV radiation into harmless heat by what is termed radiation-free deactivation. Organic substances which can be used here are, primarily, benzophenone derivatives, hydroxynaphthoquinones, phenylbenzoxazoles and phenylbenzimidazoles, digalloyl trioleate, aminobenzoic esters, salicylic esters, alicyclic dienones, cinnamic esters, benzalazine, etc. Since very high sun protection factors are not frequently achieved using organic light-protection filters, and these frequently break down under UV irradiation or demonstrate unwanted penetration properties after skin application, usually, inorganic light-absorbing pigments, in particular inorganic light-absorbing micropigments, are also added to sunscreen formulations. Examples of suitable inorganic pigments or micropigments are titanium dioxide, cerium oxide, zirconium oxide. These pigments absorb and reflect the radiation. However, in this case the micropigments frequently act as semiconductors, so that electrons are transferred from the valence band to the conductivity band. As a result of the associated electron deficit, these light-activated micropigments act as oxidants which can exhibit an action damaging to the skin. This reduces the advantageous action of customary inorganic light-absorbing micropigments. By treating the surface (calcining and/or hydrophobing with alkylsilanes), the reactive centres are marked in such a manner that photochemical processes can no longer proceed. However, the masking usually succeeds only in part, so that reactive centres still remain.

In addition, it is known that microfine TiO₂ exhibits antagonistic effects together with organic light-protection filters such as octyl methoxycinnamate. This greatly limits the use of TiO₂.

Furthermore, conventional micropigments frequently agglomerate as soon as during the formulation of the sunscreen, for example a sunscreen oil, a sunscreen milk, a sunscreen cream, a sunscreen gel, a sunscreenlotion, a sunscreen spray oil or a sunscreen spray emulsion, or after application to the skin. However, the formation of larger particles decreases their action as sunscreens and it leads, in addition, to an unwanted whitening on the skin.

It is an object of the present invention to provide inorganic light-absorbing pigments, in particular micropigments, which avoid the disadvantages of the known pigments or micropigments and, in particular, exhibit decreased oxidant activity.

We have found that this object is achieved according to the invention by using inorganic light-absorbing micropigments to which aluminium phosphates are applied in sunscreens or cosmetics emulsions.

The micropigments can be selected from all suitable inorganic light-absorbing micropigments. Preferably, they are selected from TiO₂, Ce₂O₃, ZrO₂, ZnO and mixtures thereof. Particular preference is given to using titanium dioxide (TiO₂) as micropigment.

In the micropigments the mean particle size is preferably from 5 to 100 nm, particularly preferably from 10 to 50 nm.

The aluminium phosphates can be selected from all suitable aluminium phosphates. A distinction is made here, in particular, between aluminium orthophosphate (AlPO₄), aluminium metaphosphate (Al(PO₃)₃), monoaluminium phosphate (Al(H₂PO₄)₃) and aluminium polyphosphates which are formed from Al(OH)₃ and H₃PO₄. According to the invention, in particular, aluminium orthophosphate, AlPO_{4,} is applied to the inorganic light-absorbing micropigments. For a further description of the aluminium phosphates, reference can be made to Römpp, Chemielexikon [Römpp's Chemistry Lexicon], 9th edition, Georg Thieme Verlag Stuttgart, head word “aluminium phosphates”.

The titanium dioxide can be any suitable titanium dioxide which, in particular, has been micronized or is microfine. The titanium dioxide can be untreated or pretreated (coated by calcining, chemical or physical adsorption of organic substances etc.).

Three modifications of titanium dioxide are known, anatase, brookite and rutile. According to the invention, all modifications can be used, but preference is given to anatase, rutile and mixed forms thereof.

Titanium dioxide is customarily produced by the sulphate or chloride process. Reference may be made to Römpp, Chemielexikon [Römpp's Chemistry Lexicon], 9th edition, Georg Thieme Verlag Stuttgart, head word “titanium dioxide” for a description of suitable titanium dioxides. A further description of suitable titanium dioxides is given in U.S. Pat. No. 3,981,737, U.S. Pat. No. 4,375,989, U.S. Pat. No. 5,165,995.

The titanium dioxide can be in hydrophobic form. That means that the titanium dioxide can be water-repellent on the surface. This surface treatment can comprise the pigments being provided with a thin hydrophobic layer by processes known per se. For example, the titanium dioxide can be made hydrophobic on the surface by organic silicon compounds such as alkoxysilanes. Pigments of this type are described, for example, in DE-A-33 14 742. The hydrophobic surface modification can be carried out in addition to the inventive modification.

According to the invention, the titanium dioxide is preferably treated with from 0.1 to 25% by weight, particularly preferably from 0.5 to 15% by weight, in particular from 1 to 10% by weight, of aluminium phosphates, based on the finished treated titanium dioxide.

Treatment processes are known from the prior art and are described, for example, in the abovementioned publications. The production can be carried out by precipitating aluminium phosphates from an aqueous dispersion of the inorganic light-absorbing pigments containing dissolved aluminium phosphates. Customarily, precipitation onto the pigments is carried out by changing the pH of the dispersion (e.g. TiO₂). Precipitation takes place, for example, as hydrated oxides.

It has been found according to the invention that titanium dioxide to which aluminium phosphates are applied exhibits a significantly decreased photoactivity and thus improved stability. This is found, in particular, in studies carried out using the chromametric process of the Tayca Corporation. In this process, the titanium dioxide micropigments are mixed for three minutes with butylene glycol in a weight ratio of 1:1. The mixture is then exposed to sunlight for one hour. The colouring of the butylene glycol is then studied using a Minolta Chromameter Cr-200. The higher the degree of coloration and thus the change in extinction, the greater the photoactivity of the titanium dioxide pigments.

In the study with UV light, for example, the titanium dioxide/butylene glycol paste is applied to a glass support which in turn is laid on a white card. Then the assembly is irradiated from above with UV light. For a further description of the chromametric process, reference may be made to corresponding product information from Tayca Co.

Without being limited to a theory, it is possible that the aluminium phosphates act as electrostatic spacers. The aluminium here has a calcining action, while the phosphate has a dispersing action.

In addition to the treatment with aluminium phosphates, the inorganic light-absorbing micropigments, in particular titanium dioxide pigments, can be coated with organic polymers as steric spacers. Here, any desired suitable organic polymers can be used which act as steric spacers. Particularly preferably, polyvinylpyrrolidone (PVP) and copolymers thereof are used. Corresponding copolymers are described, for example, in DE-A-199 23 672 and DE-A-199 50 089.

If a coating with organic polymers as steric separators is carried out, the amount of organic polymer is preferably from 0.1 to 10% by weight, particularly preferably from 0.5 to 3% by weight, based on the coated micropigments. Suitable coating processes are known to those skilled in the art. The coating can be carried out, for example, by introducing the micropigments into an aqueous PVP solution and subsequently drying them.

The organic polymer can have any suitable molecular weight, provided that the action as steric separator is maintained.

The inventive micropigment exhibits good spacing, which is exhibited in low bulk densities. The micropigment does not agglomerate in the applications on application to the skin or during spray-drying. Thus, for example, in sunscreens or in the cosmetics emulsions, even after application to the skin finely divided and non-agglomerated micropigments are present. Otherwise, just the agglomerates considerably impair the action of the micropigments as sunscreens.

The sunscreens or cosmetics emulsions which comprise the inventive micropigments have a good feel on the skin owing to the low primary particle sizes. In particular, no sandy feeling results when applied to the skin.

The inventive inorganic light-absorbing micropigments can thus be used advantageously in a multiplicity of sunscreens or cosmetics emulsions. The invention also relates to sunscreens or cosmetics emulsions which comprise an inorganic light-absorbing micropigment as defined above. The content of micropigments in these sunscreens or cosmetics emulsions is preferably from 1 to 40% by weight, particularly preferably from 1 to 15% by weight, based on the total sunscreen or the total cosmetic emulsion.

Sunscreens can be formulated in this case, for example, as sunscreen oil, sunscreen milk (emulsion), sunscreen cream, sunscreen gel, sunscreen lotion, sunscreen spray oil or sunscreen spray emulsion. The sunscreens or cosmetics emulsions can be present, for example, in the form of an OW, WO, PO, POW emulsion or other multiple emulsion.

The sunscreens or cosmetics emulsions can comprise other components such as cosmetically or pharmaceutically active compounds, organic water-soluble light-protection filters, other hydrophilically coated micropigments, electrolytes, glycerol, polyethylene glycol, propylene glycol, barium sulphate, alcohols, waxes, metal soaps such as magnesium stearate, Vaseline or other constituents.

For example, in addition, fragrances, fragrance oils or fragrance aromas can be added. Suitable additives are listed by way of example hereinafter.

Suitable additives are cosmetic active compounds which are, in particular, oxidation- or hydrolysis-sensitive, for example polyphenols. Those which may be mentioned here are catechins (such as epicatechin, epicatechin 3-gallate, epigallocatechin, epigallocatechin 3-gallate), flavonoids (such as luteolin, apigenin, rutin, quercitin, fisetin, kaempherol, rhametin), isoflavones (such as genistein, daidzein, glycitein, prunetin), coumarins (such as daphnetin, umbelliferone), emodin, resveratrol, oregonin.

Suitable additives are vitamins, such as retinol, tocopherol, ascorbic acid, riboflavin, pyridoxin.

Suitable additives are, in addition, overall extracts from plants which comprise, inter alia, the above molecules or classes of molecules.

The active compounds are, according to one embodiment of the invention, sunscreens. These can be present as organic light-protection filters in solid or liquid form at room temperature (25° C.). Suitable light-protection filters (UV filters) are, for example, compounds based on benzophenone, diphenyl cyanoacrylate, or p-aminobenzoic acid. Specific examples are (INCI or CTFA names) Benzophenone-3, Benzophenone-4, Benzophenone-2, Benzophenone-6, Benzophenone-9, Benzophenone-1, Benzophenone-11, Etocrylene, Octodrylene, PEG-25 PABA, Phenylbenzimidazole Sulfonic Acid, Ethylhexyl Methoxy-cinnamate, Ethylhexyl Dimethyl PABA, 4-Methylbenzylidene Camphor, Butyl Methoxydibenzoylmethane, Ethylhexyl Salicylate, Homosalate and also Methylene-bis-benzotriazolyl Tetramethylbutylphenol (2,2′-methylenebis{6-(2H-benzoetriazol-2-yl)-4-(1,1,3,3-tetramethylbutyl)phenol}, 2-hydroxy-4-methoxybenzophenone-5-sulphonic acid and 2,4,6-trianilino-p-(carbo-2′-ethylhexyl-1′-oxi)-1,3,5-triazine.

Further organic light-protection filters are Octyltriazone, Avobenzone, Octyl Methoxycinnamate, Octyl Salicylate, Benzotriazole and Triazine.

According to a further embodiment of the invention, the active compounds used are active antidandruff compounds, as are customarily present in cosmetics or pharmaceutical formulations. One example of these is Piroctone Olamine (1-hydroxy-4-methyl-6-(2,4,4-dimethylpentyl)-2(1H)pyridone; preferably in combination with 2-aminoethanol (1:1)). Other suitable compositions for treating dandruff are known to those skilled in the art.

The P/O emulsion described can also be emulsified in water or a water-in-oil emulsion. This results in a polyol-in-oil-in-water emulsion (P/O/W emulsion) which comprises at least one emulsion described and in addition at least one aqueous phase. Multiple emulsions of this type can correspond in structure to the emulsions described in DE-A-43 41 113.

When the inventive P/O emulsion is introduced into water or aqueous systems, the weight ratio of the individual phases can be varied within broad ranges. Preferably, the percentage by weight of the P/O emulsion in the final P/O/W emulsion obtained is from 0.01 to 80% by weight, particularly preferably from 0.1 to 70% by weight, in particular from 1 to 30% by weight, based on the total P/O/W emulsion.

When the inventive P/O emulsion is introduced into an O/W emulsion, the content of P/O emulsion is preferably from 0.01 to 60% by weight, particularly preferably from 0.1 to 40% by weight, in particular from 1 to 30% by weight, based on the final P/O/W emulsion obtained. In the O/W emulsion which is used for this purpose, the oil content is preferably from 1 to 80% by weight, particularly preferably from 1 to 30% by weight, based on the O/W emulsion used. Instead of a P/O emulsion, a W/O emulsion can also be introduced, which leads to a W/O/W emulsion. The individual phases of the emulsions can further have known constituents usual for the individual phases. For example, the individual phases can further comprise pharmaceutical or cosmetic active compounds which are soluble in these phases. The aqueous phase can comprise, for example, organic soluble light-protection filters, hydrophilically coated micropigment, electrolytes, alcohols, etc. Individual phases or all of the phases can also comprise solids which are preferably selected from pigments or micropigments, microspheres, silica gel and similar substances. The oil phase can comprise, for example, organically modified clay minerals, hydrophobically coated (micro)pigments, organic oil-soluble light-protection filters, oil-soluble active cosmetic compounds, waxes, metal soaps such as magnesium stearate, Vaseline or mixtures thereof. (Micro)pigments which may be mentioned are titanium dioxide, zinc oxide and barium sulphate, and also wollastonite, kaolin, talcum, Al₂O₃, bismuth oxychloride, micronized polyethylene, mica, ultramarine, eosin dyes, azo dyes. In particular, titanium dioxide or zinc oxide are customary in cosmetics as light-protection filters and may be applied particularly smoothly and uniformly to the skin by means of the inventive emulsions. Microspheres or silica gel can be used as carriers for active compounds, and waxes can be used, for example, as a base for polishes.

To produce the inventive cosmetics emulsions, emulsifiers are generally used. Examples of suitable emulsifiers are glycerol esters, polyglycerol esters, sorbitan esters, sorbitol esters, fatty alcohols, propylene glycol esters, alkyl glucoside esters, sugar esters, lecithin, silicone copolymers, lanolin and mixtures and derivatives thereof. Glycerol esters, polyglycerol esters, alkoxylates and fatty alcohols and isoalcohols can be derived, for example, from Rhizinus fatty acid, 12-hydroxy-stearic acid, isostearic acid, oleic acid, linoleic acid, linolenic acid, stearic acid, myristic acid, mauric acid and capric acid. In addition to said esters, succinates, amides or ethanolamides of the fatty acids may also be present. Fatty acid alkoxylates which can be used are, in particular, the ethoxylates, propoxylates or mixed ethoxylates/propoxylates. In addition, emulsifiers can be used which form lamellar structures. Examples of such emulsifiers are the physiological bile salts such as sodium cheolate, sodium dehydrocheolate, sodium deoxycheolate, sodium glycochealate, sodium taurochealate. Animal and plant phospholipids such as lecithins together with their hydrogenated forms and also polypeptides such as gelatin together with their modified forms can likewise be used.

Synthetic surface-active compounds which are suitable are the salts of sulphosuccinic esters, polyoxyethylene acid bethan esters, acid bethan esters and sorbitan ethers, polyoxyethylene fatty alcohol ethers, polyoxyethylene stearic esters and also corresponding mixed condensates of polyoxyethylene-methpoly-oxypropylene ethers, ethoxylated saturated glycerides, partial fatty acid glycerides and polyglycides. Examples of suitable surfactants are Biobase® ED and Ceralution.

The aqueous phase which can be used is water, aqueous solutions or mixtures of water with water-miscible liquids, such as glycerol or polyethylene glycol. In addition, electrolytes such as sodium chloride may be present in the aqueous phase. If wanted, in addition, viscosity-increasing substances or charge carriers can further be used, as are described in EP-B-0605 497.

The water phase can, furthermore, comprise propylene glycol, ethylene glycol and similar compounds and derivatives thereof.

The use of customary aids and additives in the emulsions is known to those skilled in the art.

The invention relates not only to the use of the described inorganic light-absorbing micropigments, but also to corresponding inorganic light-absorbing pigments to which aluminium phosphates are applied. The inventive coating of the micropigments also has advantages in other areas of application, in which larger pigments are used instead of micropigments. For example, in the case of dyed plastics and paper, which each contain inorganic light-absorbing pigments, the problem of decomposition under the effect of light also occurs. In these applications also the use of the inventively coated inorganic light-absorbing pigments leads to advantages. In these applications it is sufficient to apply aluminium phosphates to the pigments. The application of organic polymers as steric spacers is frequently unnecessary, since relatively large pigment particles are already present.

The invention also relates to the combination consisting of inorganic light-absorbing pigments to which AlPO₄ are applied, and organic light-protection filters, in particular octyl methoxycinnamates. In particular, the combination of microfine TiO₂ with applied AlPO₄, in contrast to conventional TiO₂ quality grades, exhibits synergistic effects. The inorganic pigments and the organic light-protection filters are preferably used in a weight ratio of 1:10 to 10:1.

Further uses for the inventive inorganic light-absorbing pigments, in particular micropigments, are known to those skilled in the art.

The invention will be described in more detail by the examples below.

### EXAMPLES

Process Steps for Producing PO4/PVP-Surface-Modified Titanium Dioxide

Pure non-doped titanium dioxide is used for surface modification, which titanium dioxide has, after preparation, first been calcined, ground and then filtered. The micropigment filter fraction is slurried in water and phosphate is added in an acidic medium. By adjusting the pH, a phosphate precipitation is carried out which is followed by a further drying process. The phosphated intermediate is then, in a further process step, admixed with an aqueous polyvinylpyrrolidone solution with stirring. The filter residue of this suspension is dried and filtered again.

The photostability of the inventive micropigments was first studied compared with known micropigments and macropigments. For this, the chromametric process described above of Tayca Corporation was used.

### Example 1

The inventive micropigment used was a titanium dioxide micropigment having a primary particle size in the range from 16 to 24 nm. The titanium dioxide pigments were coated with AlPO₄. The AlPO₄ content was about 7% by weight determined as Al₂O₃ and from 1 to 1.5% by weight determined as P₂O₅. The particles were also PVP-coated with 2.0% by weight, based on TiO2.

For purposes of comparison, different commercially conventional titanium dioxide pigments were used.

The pigments were mixed in a mass ratio of 1:2 with butylene glycol for three minutes. The resultant paste was exposed to a UV light source for one hour. The distance between the sample and the UV light source was 30 cm.

Before and after irradiation, the extinction was determined using a Minolta Chromameter CR-300. The degree of discoloration was then determined, a large numerical value indicating a high degree of discoloration. The discoloration is a direct measure of the photoactivity of the titanium dioxide particles. Photostable titanium dioxide particles lead to a low discoloration in the titanium dioxide/butylene glycol paste.

The results are summarized in the table below:

| Pigment type | Manufacturer | Degree of discoloration |
|---|---|---|
| According to the | Kemira | 2.90 |
| invention | | |
| UV-titanium M262 | Kemira | 8.26 |
| UV-titanium M212 | Kemira | 9.05 |
| Cardre TiO2-Si2 | Cardre | 14.11 |
| Cardre TiO2-AS | Cardre | 15.27 |
| Eusolex T 2000 | Sachtleben | 28.90 |
| Tayca MT100T | Tayca Corp. | 48.77 |

### Example 2

Differing titanium dioxide micropigments and micropigments and macropigments known from the prior art were dispersed in alkyl benzoate. The inventive micropigment used was the micropigment described in Example 1. In each case 10% by weight titanium dioxide was dispersed in alkyl benzoate which contained 1% by weight of ascorbyl palmitate. The percentages by weight relate to the finished composition. As a reference, a mixture of 10% titanium dioxide, dispersed in alkyl benzoate, was used. Each dispersion was mixed for one minute.

Measurement with the Minolta Chromameter CR-300 and irradiation were carried out as described in Example 1. The results are summarized in the table below:

| Pigment quality | Manufacturer | Degree of discoloration |
|---|---|---|
| According to the invention | Kemira | 0.71 |
| UV-titanium M262 | Kemira | 1.10 |
| UV-titanium M212 | Kemira | 1.28 |
| Cardre TiO2-AS | Cardre | 4.00 |
| Eusolex T 2000 | Sachtleben | 4.78 |
| Tayca MT100T | Tayca Corp. | 20.40 |

### Example 3

Inventive micropigments having differing amounts of AlPO₄ were studied for their photostability and their degree of discoloration was determined over time using the Chromameter. The following pigments were used

Time [h]:

| | 0.0166 | 1 | 18 | 120 |
|---|---|---|---|---|
| SAMPLE 7/1 | 0.71 | 1.65 | 1.91 | 3.09 |
| SAMPLE 7/2 | 0.95 | 1.88 | 2.8 | 3.31 |
| SAMPLE 7/6 | 2.91 | 5.63 | 7.53 | 8.07 |
| SAMPLE 7/7 | 1.50 | 2.45 | 4.06 | 5.4 |

Sample 7/1 contained 1.2% of P₂O₅, Sample 7/2 contained 2.4% P₂O₅. Both samples contained 2% PVP.

### Example 4

The following example demonstrates the synergistic effect of microfine TiO₂ onto which AlPO₄ was coated in combination with PVP (SPF=Sun Protection Factor).

| | | |
|---|---|---|
| in vivo SPF: | 20.2 ± 2.9 | 11.2 ± 1.2 |
| ratio SPF/UV filter: | 1.5 | 0.83 |

| Trade name | Supplier | CTFA/INCI | OW-1-3/0 [% by wt.] | OW-2-3/0 [% by wt.] |
|---|---|---|---|---|
| Phase A | | | | |
| Ceralution H | Sasol | Behenyl alcohol, glyceryl | 5.00 | 5.00 |
| | | stearate, glyceryl stearate/ | | |
| | | citrate, sodium dicocoyl- | | |
| | | ethylenediamine PEG-15 | | |
| | | sulphate | | |
| Cosmacol ECI | Sasol | Tri-C12-13 alkyl citrate | 2.00 | 2.00 |
| | Augusta | | | |
| | S.p.A. | | | |
| Cetiol B | Cognis | Dibutyl adipate | 3.00 | 3.00 |
| Neo Heliopan AV | H&R | Ethylhexyl methoxy- | 7.50 | 7.50 |
| | | cinnamate | | |
| IPP | Cognis | Isopropyl palmitate | 4.00 | 4.00 |
| Vitamin E acetate | Roche | Tocopheryl acetate | 0.50 | 0.50 |
| Sample 7/1 | Kemira | | 6.00 | 0.00 |
| UV-titanium M | Kemira | Titanium dioxide, alumina, | 0.00 | 6.00 |
| 262 | | dimethicone | | |
| Phase B | | | | |
| Demin. water | — | Aqua | 62.70 | 62.70 |
| Ceralution F | Sasol | Sodium lauroyl lactylate, | 1.00 | 1.00 |
| | | sodium dicocoylethylene- | | |
| | | diamine PEG-15 sulphate | | |
| Pricerine 9091 | Uniquema | Glycerin | 4.00 | 4.00 |
| Hydrolite-5 | Cosnaderm | Pentylene glycol | 3.00 | 3.00 |
| Keltrol | Kelco | Xanthan gum | 0.30 | 0.30 |
| Panthenol | Hoffmann | Panthenol | 1.00 | 1.00 |
| | LaRoche | | | |
| Total: | | | 100.00 | 100.00 |

Production

To produce the sunscreen formulation, Phases A and B were heated separately to from 60 to 70° C. Phase A was then added to Phase B, and the mixture was homogenized for two hours. The mixture was then cooled to 40° C. and was homogenized for a further two minutes.

1-11. (canceled)

12. Sunscreen or cosmetics emulsion comprising an inorganic light-absorbing micropigments to which aluminium phosphates are applied with or without organic light-protection filters.

13. Sunscreen or cosmetic emulsion according to claim 12, characterized in that the inorganic light-absorbing micropigment is selected from TiO₂, Ce₂O₃, ZrO₂, ZnO and mixtures thereof.

14. Sunscreen or cosmetic emulsion according to claim 12, characterized in that the mean particle size in the inorganic light-absorbing micropigment is from 5 to 100 nm.

15. Sunscreen or cosmetic emulsion according to claim 12, characterized in that the inorganic light-absorbing micropigment is additionally coated with organic polymers as steric spacers.

16. Sunscreen or cosmetic emulsion according to claim 12, characterized in that the sunscreens or cosmetics emulsions are present in the form of an OW, WO, PO, POW emulsion or other multiple emulsion.

17. Inorganic light-absorbing micro-pigment to which aluminium phosphates are applied characterized in that the mean particle size in the inorganic light-absorbing micropigment is from 5 to 100 nm.

18. Micropigment according to claim 17, characterized in that the mean particle size is from 10 to 50 nm.

19. Micropigment according to claim 17, characterized in that it is additionally coated with organic polymers as steric spacers.

20. Process for producing in organic light-absorbing micropigments according to claim 17 by precipitating aluminium phosphates from an aqueous dispersion of the inorganic light-absorbing micropigment which contains dissolved aluminium phosphates.
